(19) United States
(12) Patent Application Publication
(10) Pub. No.: US 2024/0066087 A1
(43) Pub. Date: Feb. 29, 2024

(54) SUTHERLANDIA EXTRACT AND THE USE THEREOF IN THE MANUFACTURE OF A MEDICAMENT

(71) Applicant: ACUPHARM INTERNATIONAL PROPRIETARY LIMITED, Gaborone (BW)

(72) Inventors: Johannes Francois MARAIS, Potchefstroom (ZA); Jacobus Johannes VAN JAARSVELD, Potchefstroom (ZA); James Clive Hurwitz LAPORTA, Cape Town (ZA)

(21) Appl. No.: 18/268,232

(22) PCT Filed: Dec. 15, 2021
(86) PCT No.: PCT/IB2021/061739

§ 371 Date: Jun. 18, 2023

(30) Foreign Application Priority Data
Dec. 18, 2020 (NL) 2027158

(51) Int. Cl. A61K 36/48; A61P 35/00

(52) U.S. Cl. CPC A61K 36/48; A61P 35/00; A61K 2236/333

(57) ABSTRACT

The present invention relates to an extract of the plant genus Sutherlandia, the method comprising dispersing Sutherlandia plant material in ethanol solution adjusting the pH of the solution and storing the adjusted solution, re-adjusting the pH of the solution, storing the re-adjusted solution and separating and filtering the solution to retain the supernatant. The invention further relates to the use of such a method in the manufacture of a medicament for the treatment of cancer.

### FIELD OF APPLICATION OF THE INVENTION

The present invention relates to an extract of the plant genus Sutherlandia frutescens. The invention further relates to the use thereof in the manufacture of a medicament.

### BACKGROUND TO THE INVENTION

Sutherlandia frutescens (“Sutherlandia”) is an indigenous medicinal plant extensively used in South Africa to treat a variety of health conditions. It is a fairly widespread, drought-resistant plant that grows in the Western, Eastern, and Northern Cape provinces, some areas of KwaZulu-Natal and Botswana, varying in its chemical and genetic makeup across these geographic areas. Sutherlandia extracts are known to induce apoptosis in cultured carcinoma cells in in vitro studies. Sutherlandia extract treatment is further known to induce apoptosis and modulate the PI3-kinase pathway in colon cancer cells in vitro. A number of different preparations of Sutherlandia are known to be made with water or alcohol; however their application in the treatment of cancer is limited and the specific compounds and isolates that may be of value in cancer treatment—such as curcumin from turmeric—have not been discovered.

A known extraction method for obtaining an extract of Sutherlandia is described by Tai et al (In vitro culture studies of Sutherlandia frutescens on human tumor cell lines. Tai J, Cheung S, Chan E, Hasman D. J Ethnopharmacol. 2004 July; 93(1):9-19). This process uses tablets of powdered Sutherlandia material obtained commercially, and is intended for bench-top extraction of Sutherlandia phytochemicals for use in research.

Further known processes for producing extracts of Sutherlandia are those well known in the art for the production of plant essential oils and other plant-derived products. These include steam distillation, maceration, solvent extraction (of which tinctures are a subset) and supercritical CO2 extraction. In the process of steam distillation, steam is used to carry plant products (principally oils) up a distillation column, with the steam and the oil then captured by cooling and separated into polar (hydrolat) and non-polar fractions (essential oil) fractions. In the process of maceration, a solvent (in the form of heated oil) is used to carry the non-polar fraction. In the process of solvent extraction, a solvent (usually an alcohol or non-polar compound) is brought into contact with a plant sample in order to absorb a portion of its phytocompounds. The compounds which are absorbed will depend principally on their polarity, which affects the rate and absolute concentration with which they enter the solvent. Numerous solvents, with varying polarities, are known as demonstrated in the table herein below.

| | | Boiling | Melting | | |
|---|---|---|---|---|---|
| | | Point | Point | Density | Relative |
| Solvent | Formula | (° C.) | (° C.) | (g/ml) | Polarity |
| cyclohexane | C₆H₁₂ | 80.7 | 6.6 | 0.779 | 0.006 |
| pentane | C₅H₁₂ | 36.1 | −129.7 | 0.626 | 0.009 |
| hexane | C₆H₁₄ | 69 | −95 | 0.655 | 0.009 |
| heptane | C₇H₁₆ | 98 | −90.6 | 0.684 | 0.012 |
| carbon tetrachloride | CCl₄ | 76.7 | −22.4 | 1.594 | 0.052 |
| carbon disulfide | CS₂ | 46.3 | −111.6 | 1.263 | 0.065 |
| p-xylene | C₈H₁₀ | 138.3 | 13.3 | 0.861 | 0.074 |
| toluene | C₇H₈ | 110.6 | −93 | 0.867 | 0.099 |
| benzene | C₆H₆ | 80.1 | 5.5 | 0.879 | 0.111 |
| ether | C₄H₁₀O | 34.6 | −116.3 | 0.713 | 0.117 |
| methyl t-butyl ether | C₅H₁₂O | 55.2 | −109 | 0.741 | 0.124 |
| (MTBE) | | | | | |
| diethylamine | C₄H₁₁N | 56.3 | −48 | 0.706 | 0.145 |
| dioxane | C₄H₈O₂ | 101.1 | 11.8 | 1.033 | 0.164 |
| N,N-dimethylaniline | C₈H₁₁N | 194.2 | 2.4 | 0.956 | 0.179 |
| chlorobenzene | C₆H₅Cl | 132 | −45.6 | 1.106 | 0.188 |
| anisole | C₇H₈O | 153.7 | −37.5 | 0.996 | 0.198 |
| tetrahydrofuran (THF) | C₄H₈O | 66 | −108.4 | 0.886 | 0.207 |
| ethyl acetate | C₄H₈O₂ | 77 | −83.6 | 0.894 | 0.228 |
| ethyl benzoate | C₉H₁₀O₂ | 213 | −34.6 | 1.047 | 0.228 |
| dimethoxyethane | C₄H₁₀O₂ | 85 | −58 | 0.868 | 0.231 |
| (glyme) | | | | | |
| diglyme | C₆H₁₄O₃ | 162 | −64 | 0.945 | 0.244 |
| methyl acetate | C₃H₆O₂ | 56.9 | −98.1 | 0.933 | 0.253 |
| chloroform | CHCl₃ | 61.2 | −63.5 | 1.498 | 0.259 |
| 3-pentanone | C₅H₁₂O | 101.7 | −39.8 | 0.814 | 0.265 |
| 1,1-dichloroethane | C₂H₄Cl₂ | 57.3 | −97.0 | 1.176 | 0.269 |
| di-n-butyl phthalate | C₁₆H₂₂O₄ | 340 | −35 | 1.049 | 0.272 |
| cyclohexanone | C₆H₁₀O | 155.6 | −16.4 | 0.948 | 0.281 |
| pyridine | C₅H₅N | 115.5 | −42 | 0.982 | 0.302 |
| dimethylphthalate | C₁₀H₁₀O₄ | 283.8 | 1 | 1.190 | 0.309 |
| methylene chloride | CH₂Cl₂ | 39.8 | −96.7 | 1.326 | 0.309 |
| 2-pentanone | C₅H₁₀O | 102.3 | −76.9 | 0.809 | 0.321 |
| 2-butanone | C₄H₈O | 79.6 | −86.3 | 0.805 | 0.327 |
| 1,2-dichloroethane | C₂H₄Cl₂ | 83.5 | −35.4 | 1.235 | 0.327 |
| benzonitrile | C₇H₅N | 205 | −13 | 0.996 | 0.333 |
| acetone | C₃H₆O | 56.2 | −94.3 | 0.786 | 0.355 |
| dimethylformamide | C₃H₇NO | 153 | −61 | 0.944 | 0.386 |
| (DMF) | | | | | |
| t-butyl alcohol | C₄H₁₀O | 82.2 | 25.5 | 0.786 | 0.389 |
| aniline | C₆H₇N | 184.4 | −6.0 | 1.022 | 0.420 |
| dimethylsulfoxide | C₂H₆OS | 189 | 18.4 | 1.092 | 0.444 |
| (DMSO) | | | | | |
| acetonitrile | C₂H₃N | 81.6 | −46 | 0.786 | 0.460 |
| 3-pentanol | C₅H₁₂O | 115.3 | −8 | 0.821 | 0.463 |
| 2-pentanol | C₅H₁₂O | 119.0 | −50 | 0.810 | 0.488 |
| 2-butanol | C₄H₁₀O | 99.5 | −114.7 | 0.808 | 0.506 |
| cyclohexanol | C₆H₁₂O | 161.1 | 25.2 | 0.962 | 0.509 |
| 1-octanol | C₈H₁₈O | 194.4 | −15 | 0.827 | 0.537 |
| 2-propanol | C₃H₈O | 82.4 | −88.5 | 0.785 | 0.546 |
| 1-heptanol | C₇H₁₆O | 176.4 | −35 | 0.819 | 0.549 |
| i-butanol | C₄H₁₀O | 107.9 | −108.2 | 0.803 | 0.552 |
| 1-hexanol | C₆H₁₄O | 158 | −46.7 | 0.814 | 0.559 |
| 1-pentanol | C₅H₁₂O | 138.0 | −78.2 | 0.814 | 0.568 |
| acetyl acetone | C₅H₈O₂ | 140.4 | −23 | 0.975 | 0.571 |
| ethyl acetoacetate | C₆H₁₀O₃ | 180.4 | −80 | 1.028 | 0.577 |
| 1-butanol | C₄H₁₀O | 117.6 | −89.5 | 0.81 | 0.586 |
| benzyl alcohol | C₇H₈O | 205.4 | −15.3 | 1.042 | 0.608 |
| 1-propanol | C₃H₈O | 97 | −126 | 0.803 | 0.617 |
| Acetic acid | C₂H4O₂ | 118 | 16.6 | 1.049 | 0.648 |
| 2-aminoethanol | C₂H₇NO | 170.9 | 10.5 | 1.018 | 0.651 |
| ethanol | C₂H₆O | 78.5 | −114.1 | 0.789 | 0.654 |
| diethylene glycol | C₄H₁₀O₃ | 245 | −10 | 1.118 | 0.713 |
| methanol | CH₄O | 64.6 | −98 | 0.791 | 0.762 |
| ethylene glycol | C₂H₆O₂ | 197 | −13 | 1.115 | 0.790 |
| glycerin | C₃H₈O₃ | 290 | 17.8 | 1.261 | 0.812 |
| water, heavy | D₂O | 101.3 | 4 | 1.107 | 0.991 |

Following contact with the plant material, the solvent mixture is then referred to as an extract. This extract is then distilled at a low pressure, reducing the boiling point of the solvent, so that only gentle heat is required to remove it and leaving behind the aromatic molecules (the concentrated extract). The concentrated extract is then cooled, causing it to solidify to a waxy solid, which is then ‘washed’ and warmed in alcohol. The final, washed product is then chilled to separate out any remaining waxes, filtered and the alcohol removed by vacuum distillation at the lowest possible temperature to produce a final product.

### OBJECT OF THE INVENTION

It is accordingly an object of the present invention to provide a method of extraction of Sutherlandia, an extract of Sutherlandia or derivatives and isolates thereof, and a novel medicament derived therefrom, which at least partially ameliorates or overcomes the disadvantages of the prior art.

### SUMMARY OF THE INVENTION

According to a first aspect of the invention, there is provided a method for obtaining an aqueously-soluble extract of Sutherlandia frutescens (“Sutherlandia”), the method comprising:
dispersing Sutherlandia plant material in ethanol solution with a concentration of at least 70% v/v;
adjusting the pH of the solution to at least 10;
storing the adjusted solution for at least 24 hours;
re-adjusting the solution to a pH of 7;
storing the re-adjusted solution for at least 24 hours;
separating the supernatant from the sediment, retaining the supernatant; and
filtering the supernatant.

In an embodiment of the invention, the ratio of ethanol solution to Sutherlandia plant material may be at least 5 ml per gram of extract. In a preferred embodiment of the invention, the ratio of ethanol solution to Sutherlandia plant material may be 10 ml per gram of extract.

According to the invention, the pH of the solution is adjusted to a pH of 12. In an embodiment of the invention, the pH is adjusted using sodium hydroxide. The pH of the solution may be re-adjusted using hydrochloric acid.

In terms of the invention, the adjusted solution may be stored for at least five days. Preferably, the adjusted solution may be stored for ten days.

In an embodiment of the invention, the re-adjusted solution may be stored for at least five days. Preferably, the re-adjusted solution may be stored for at least ten days.

In terms of the invention, the adjusted solution may be mixed daily during storage. The re-adjusted solution may be mixed for at most the first 50% of the storage period and then left to settle for the remaining period.

According to an embodiment of the first aspect of the invention, the Sutherlandia plant material is leaf material. According to an alternative embodiment of the first aspect of the invention, the Sutherlandia plant material is root material. According to another alternative embodiment of the first aspect of the invention, the Sutherlandia plant material is stem material.

In terms of the invention, the method further includes addition of a solubiliser. In an embodiment of the invention, the solubiliser may be Kolliphor® HS 15 and/or Kollidon® 25.

The invention provides for the extract to be processed further after extraction to isolate an active fraction. In terms of the invention, the active fraction is isolated to obtain one or more active compounds.

The invention yet further provides for the extract, active fraction or active compound/s to be used as a component of a composition.

According to a second aspect of the invention, there is provided a composition derived from the method as described according to the first aspect of the invention. The composition may be used for the treatment of cancer.

Here ‘cancer’ may include, but is not limited to, myeloma, amyloidosis, solitary plasmacytoma, anaplastic large cell lymphoma, Burkitt's lymphoma, Cutaneous T-Cell Lymphoma, diffuse large B-Cell lymphoma, follicular lymphoma, lymphoblastic lymphoma, MALT lymphoma, mantle cell lymphoma, primary mediastinal large B-Cell lymphoma, nodal marginal zone B-Cell lymphoma, small lymphocytic lymphoma, thyroid lymphoma, Waldenstrom's macroglobulinaemia, hairy cell leukaemia, essential thrombocythaemia, myelofibrosis, polycythaemia vera, Monoclonal Gammopathy of Unknown Significance (MGUS), myleodysplatic syndromes, chondrosarcoma, ewing's sarcoma, malignant fibrous histiocytoma of bone or osteosarcoma, osteosarcoma, rhabdomyosarcoma, heart cancer, astrocytoma, brainstem glioma, pilocytic astrocytoma, ependymoma, primitive neuroectodermal tumour, cerebellar astrocytoma, cerebral astrocytoma, glioma, medulloblastoma, neuroblastoma, oligodendroglioma, pineal astrocytoma, pituitary adenoma, visual pathway and hypothalamic glioma, breast cancer, invasive lobular carcinoma, tubular carcinoma, invasive cribriform carcinoma, medullary carcinoma, male breast cancer, phyllodes tumour, inflammatory breast cancer, adrenocortical carcinoma, islet cell carcinoma, multiple endocrine neoplasia syndrome, parathyroid cancer, pheochromocytoma, thyroid cancer, merkel cell carcinoma, uveal melanoma, retinoblastoma, anal cancer, appendix cancer, cholangiocarcinoma, carcinoid tumour, colon cancer, extrahepatic bile duct cancer, gallbladder cancer, gastric (stomach) cancer, gastrointestinal carcinoid tumour, gastrointestinal stromal tumour, hepatocellular cancer, islet cell pancreatic cancer, rectal cancer, bladder cancer, cervical cancer, endometrial cancer, extragonadal germ cell tumour, ovarian cancer, ovarian epithelial cancer, ovarian germ cell tumour, penile cancer, renal cell carcinoma, transitional cell cancer (renal pelvis and ureter), prostate cancer, testicular cancer, gestational trophoblastic tumour, transitional cell cancer (ureter and renal pelvis), urethral cancer, uterine sarcoma, vaginal cancer, vulvar cancer, wilms tumour, esophageal cancer, nasopharyngeal carcinoma, oral cancer, oropharyngeal cancer, paranasal sinus and nasal cavity cancer, pharyngeal cancer, salivary gland cancer, hypopharyngeal cancer, acute biphenotypic leukaemia, acute eosinophilic leukaemia, acute lymphoblastic leukaemia, acute myeloid leukaemia, acute myeloid dendritic cell leukaemia, AIDS-related lymphoma, anaplastic large cell lymphoma, angioimmunoblastic T-cell lymphoma, B-cell prolymphocytic leukaemia, chronic lymphocytic leukaemia, chronic myelogenous leukaemia, cutaneous T-cell lymphoma, diffuse large B-cell lymphoma, follicular lymphoma, hairy cell leukaemia, hepatosplenic T-cell lymphoma, Hodgkin's lymphoma, hairy cell leukaemia, intravascular large B-cell lymphoma, large granular lymphocytic leukaemia, lymphoplasmacytic lymphoma, lymphomatoid granulomatosis, mantle cell lymphoma, marginal zone B-cell lymphoma, mast cell leukaemia, mediastinal large B cell lymphoma, multiple myeloma/plasma cell neoplasm, myelodysplastic syndromes, mucosa-associated lymphoid tissue lymphoma, mycosis fungoides, nodal marginal zone B cell lymphoma, non-Hodgkin lymphoma, precursor B lymphoblastic leukaemia, primary central nervous system lymphoma, primary cutaneous follicular lymphoma, primary cutaneous immunocytoma, primary effusion lymphoma, plasmablastic lymphoma, Sézary syndrome, splenic marginal zone lymphoma, T-cell prolymphocytic leukaemia, basal cell carcinoma, squamous cell carcinoma, skin adnexal tumour, melanoma, Merkel cell carcinoma, sarcomas of primary cutaneous origin, lymphomas of primary cutaneous origin, bronchial adenomas, bronchial carcinoids, small cell lung cancer, mesothelioma, non-small cell lung cancer, pleuropulmonary blastoma, laryngeal cancer, thymoma and thymic carcinoma, Kaposi sarcoma, epithelioid hemangioendothelioma, desmoplastic small round cell tumour, and liposarcoma. In terms of the invention, the composition is dried. Preferably, the drying is carried out in combination with a liposomal compound.

In an embodiment of the invention, the composition may be further formulated for oral administration using capsules.

In an alternative embodiment of the invention, the composition may be formulated for intravenous (IV) injection.

In a further embodiment of the invention, the composition may be formulated for administration as a cream.

In a yet further embodiment of the invention, the composition may be formulated by dilution into a buffered solution.

In terms of the invention, the composition further comprises a known anti-cancer compound.

Here known anti-cancer compounds may include (but are not limited to) cyclophosphamide, mechlorethamine, chlorambucil, melphalan, dacarbazine, temozolomide, anthracyclines, daunorubicin, doxorubicin, epirubicin, idarubicin, mitoxantrone, valrubicin, paclitaxel, docetaxel, abraxane, taxotere, epothilones, vorinostat, romidepsin, irinotecan, topotecan, etoposide, teniposide, tafluposide, bortezomib, erlotinib, gefitinib, imatinib, vemurafenib, vismodegib, azacitidine, 5-fluorouracil, azathioprine, 6-mercaptopurine, capecitabine, capecitabine, cytarabine, cytarabine, doxifluridine, floxuridine, fluorouracil, fludarabine, gemcitabine, gemcitabine, hydroxyurea, hydroxycarbamide, mercaptopurine, methotrexate, methotrexate, pemetrexed, tioguanine, phototrexate, bleomycin, actinomycin, carboplatin, cisplatin, oxaliplatin, tretinoin, alitretinoin, bexarotene, vinblastine, vincristine, vindesine, vinorelbine, ado-trastuzumab emtansine, afatinib, aldesleukin, alectinib, alemtuzumab, atezolizumab, avelumab, axitinib, belimumab, belinostat, bevacizumab, blinatumomab, bortezomib, bosutinib, brentuximab vedotin, brigatinib, cabozantinib, canakinumab, carfilzomib, ceritinib, cetuximab, cobimetinib, crizotinib, dabrafenib, daratumumab, dasatinib, denosumab, dinutuximab, durvalumab, elotuzumab, enasidenib, erlotinib, everolimus, febendazole, gefitinib, ibritumomab tiuxetan, ibrutinib, idelalisib, imatinib, ipilimumab, ixazomib, lapatinib, lenvatinib, midostaurin, necitumumab, neratinib, nilotinib, niraparib, nivolumab, obinutuzumab, ofatumumab, olaparib, olaratumab, osimertinib, palbociclib, panitumumab, panobinostat, pazopanib, pembrolizumab, pertuzumab, ponatinib, ramucirumab, regorafenib, ribociclib, rituximab, rituximab/hyaluronidase human, romidepsin, rucaparib, ruxolitinib, siltuximab, sipuleucel-t, sonidegib, sorafenib, temsirolimus, tocilizumab, tofacitinib, tositumomab, trametinib, trastuzumab, vandetanib, vemurafenib, venetoclax, vismodegib, vorinostat, ziv-aflibercept, Tumor-Infiltrating Lymphocyte (TIL) therapy, Engineered T Cell Receptor (TCR) therapy, Chimeric Antigen Receptor (CAR) T cell therapy, natural killer (NK) cell therapy, axicabtagene ciloleucel, brexucabtagene autoleucel, tisagenlecleucel, indocyanin green, chlorin e6, Methylene blue, ALA, photosense, and combinations thereof.

In terms of the invention, the composition further comprises a known natural or naturally-derived compound.

Here known natural or naturally-derived compounds may include, but are not limited to, aloe, Artemesia absinthium, Artemesia affra, Artemesia aulgaris, Artemesinin, Artemsia annua, Artesunat, Artesunate, ascorbic acid, berberine, Bidens pilosa, bioperine, boswellia, Genieila asiatica, Cnicus Benedictus, Cordyceps spp., Drosera capensis, Ganoderma spp., Genesteine, gingerol, ginseng, ginsenoside, ginsenosoides, green rooibos, honeybush broad leaf, Hypoestes aristate, Hypoxis haemor, indirubin, Leonotis leonurus, Mimosa pudicca, moringa, Nigella sativa, olive leaf extract, oridonin, oxymutrine, proancocydin, quercetin, resveratrol, sutherak, tanhinone, tanshins, thin leaf honeybush, thymoquinone, ursolic acid, vitamin C, vitamin K3, xalpalthinin, SAC-Calcium and combinations thereof.

In an embodiment of the invention, the composition may be formulated by drying.

According to a third aspect of the invention, there is provided a method of treating cancer, comprising administering the composition, substantially as herein described according to the second aspect of the invention, to a subject in need thereof.

According to a fourth aspect of the invention, there is provided for use of the composition, substantially as herein described according to the second aspect of the invention, in the manufacture of a medicament for the treatment of cancer.

The above and other characteristics, features and advantages of the present invention will become apparent from the following detailed description, taken in conjunction with the accompanying drawing which illustrate, by way of example, the principles of the invention. This description is given for the sake of example only, without limiting the scope of the invention. The reference figures quoted below refer to the attached drawings.

### BRIEF DESCRIPTION OF THE DRAWINGS

One embodiment of the invention is described below, by way of a non-limiting example only, and with reference to the accompanying drawings in which:

FIG. 1 is a graph illustrating the efficacy of a control sample vinorelbine;

FIG. 2 is a graph illustrating the efficacy of a control sample docetaxel;

FIG. 3 is a graph illustrating the efficacy of a control sample paclitaxel;

FIG. 4 is a graph illustrating the efficacy of a control sample vincristine;

FIG. 5 is a graph illustrating the efficacy of a control sample mitoxantrone;

FIG. 6 is a graph illustrating the efficacy of a control sample vorinostat;

FIG. 7 is a graph illustrating the efficacy of a control sample clofarabine;

FIG. 8 is a graph illustrating the efficacy of a control sample cediranib;

FIG. 9 is a graph illustrating the efficacy of a control sample axitinib;

FIG. 10 is a graph illustrating the efficacy of a control sample dasatinib;

FIG. 11 is a graph illustrating the efficacy of a control sample gefitinib;

FIG. 12 is a graph illustrating the efficacy of a control sample ponatinib;

FIG. 13 is a graph illustrating the efficacy of a control sample nintedanib; and

FIG. 14 is a graph illustrating the efficacy of a preferred embodiment of the invention;

The presently disclosed subject matter will now be described more fully hereinafter with reference to the accompanying Example, in which representative embodiments are shown. The presently disclosed subject matter can, however, be embodied in different forms and should not be construed as limited to the embodiments set forth herein. Rather, these embodiments are provided so that this disclosure will be thorough and complete, and will fully convey the scope of the embodiments to those skilled in the art.

### DESCRIPTION OF PREFERRED EMBODIMENTS OF THE INVENTION

A non-limiting example of a preferred embodiment of the invention is described in more detail below, with reference to the Figures.

### Example 1

Sutherlandia Extraction and Processing

In order to obtain an aqueously-soluble Sutherlandia extract 100 g of a commercially-available 95% Sutherlandia powder extract combined with 500 ml of 90% ethanol diluted from absolute alcohol using deionised water. This solution was then stirred or shaken until all the powder was dispersed in the solution. In a separate container a 25% Sodium Hydroxide solution was then prepared, which was used to adjust the pH of the solution to 12. After adjustment the solution was then stored at room temperature for 10 days, being mixed every day. Following storage, the solution was re-adjusted to a pH of 7 using HCl and stored for another ten days at room temperature. The re-adjusted solution was mixed daily for the first five days, then left to settle. Thereafter the solution was separated from the sediment and filtered, before being sent for HPLC analysis.

Here the use of mixture and settling rather than sonication and filtration appears to be important in obtaining the favourable results seen in vitro.

### Example 2

Comparative studies were performed using Sutherlandia leaf extract in water (referred to as ACPJL35), Sutherlandia leaf extract in 50% ethanol and water solution (referred to as ACPJL28), and Sutherlandia leaf extract in 90% ethanol and water solution (referred to as ACPJL07). These extracts were compared to a number of known botanical isolates with anti-cancer activity, as well as a number of known anti-cancer drugs, using a dose response design with cumulative cell death as the dependent variable. These studies were conducted on in vitro samples of cultured tissue, taken as biopsy samples from patients as part of a standard culture and testing process done in order to optimise treatment.

A representative sample of such a study is provided herein below.

Summary of Study of Patient with Assigned Number 19055

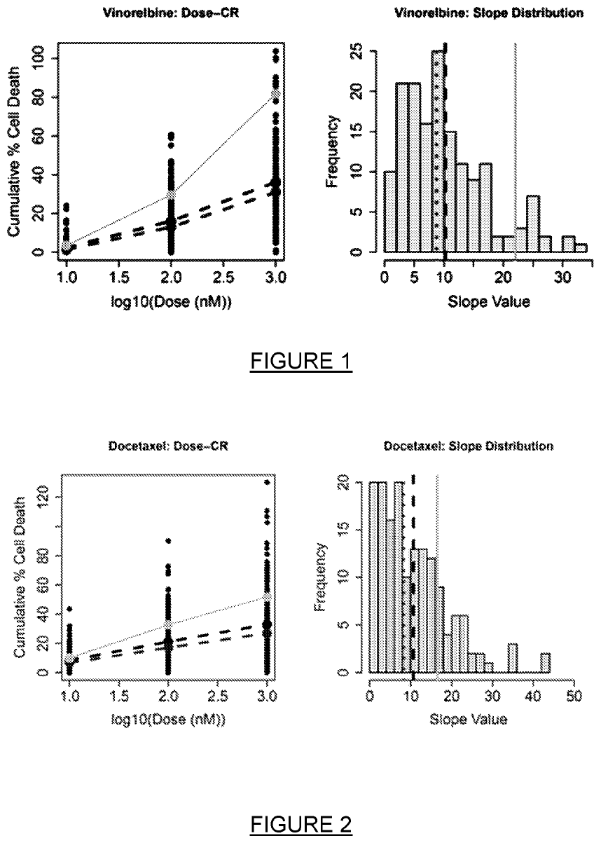

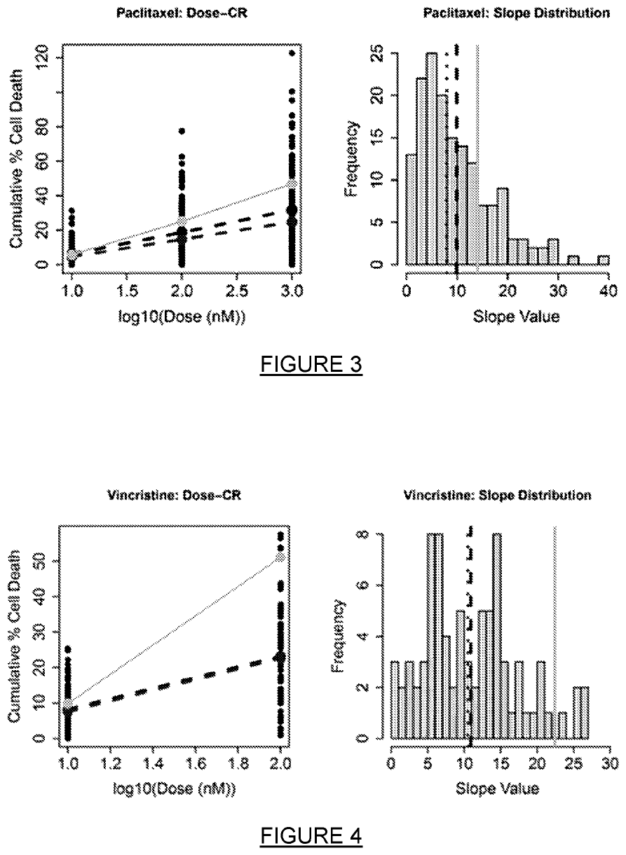

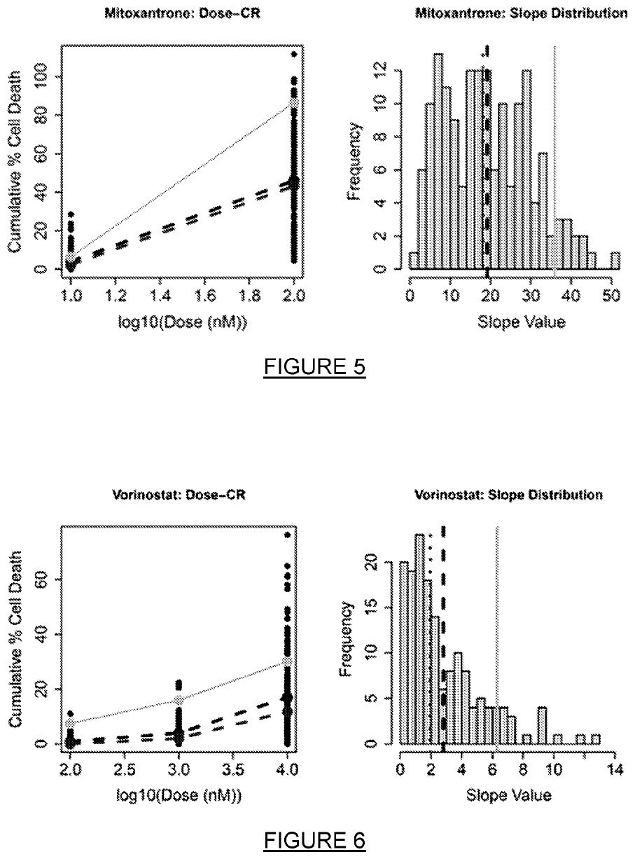

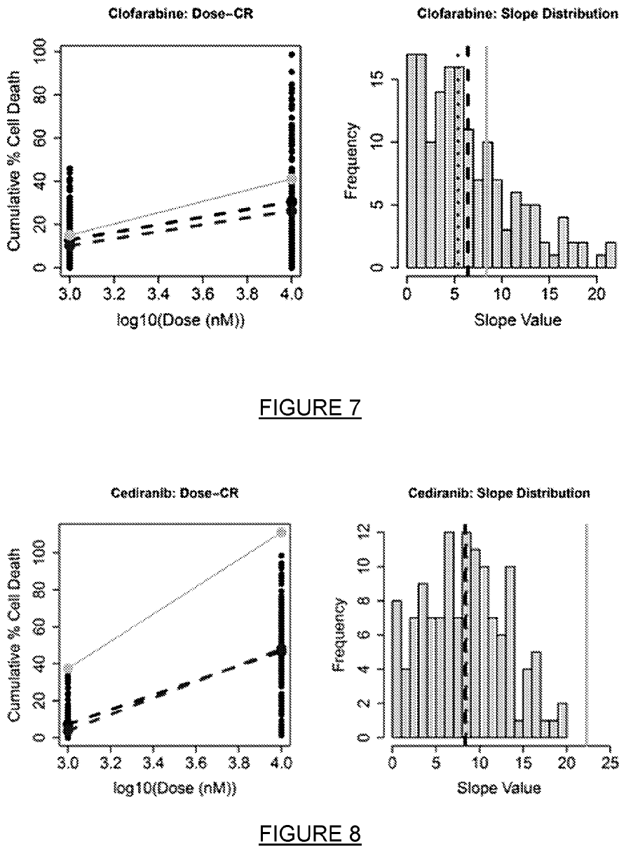

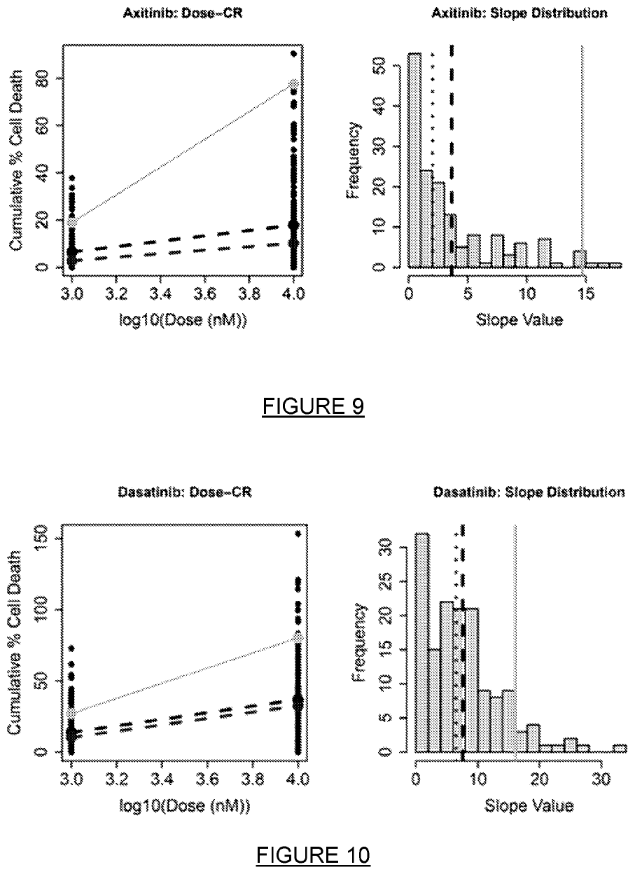

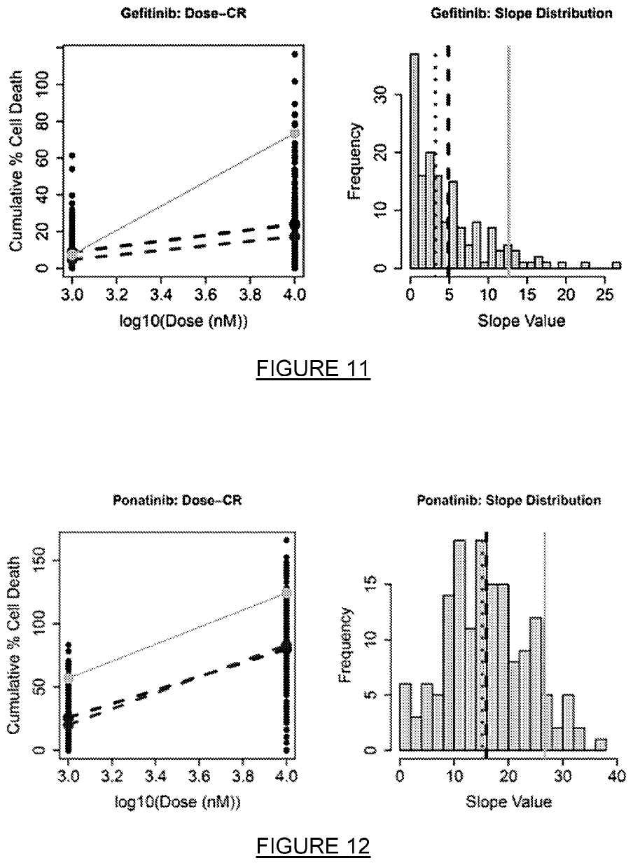

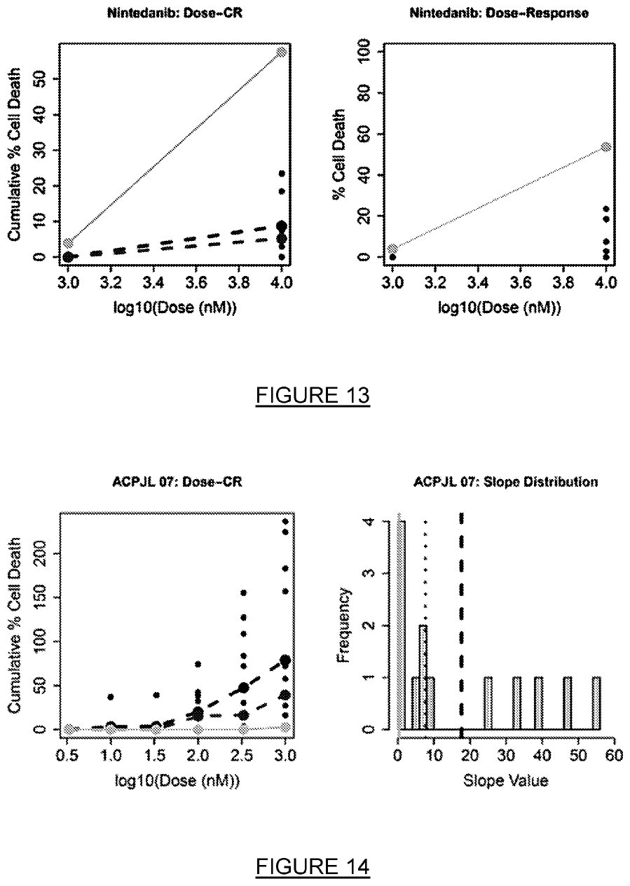

Patient from whom sample was obtained was a 70-year-old female presenting with a diagnosis of Breast DCIS papillary neoplasm. The sample was obtained via needle biopsy, before being cultured. As part of the standard testing process a number of non-targeted drugs (Vinorelbine, Docetaxel, Paclitaxel, Vincristine, Mitoxantrone, Vorinostat, Clofarabine, the dose-CR curves and slope distributions for which can be seen in FIGS. 1 to 6 and targeted drugs (Cediranib, Axitinib, Dasatinib, Gefitinib, Ponatinib, the dose-CR curves and slope distributions for which can be seen in FIGS. 7 to 12) were identified as having potential benefit. Another drug, Nintedanib (FIG. 13), was identified as a new drug having good efficacy and a positive dose response curve.

Of the botanicals tested, ACPJL 07 (the dose-CR curves and slope distributions for which can be seen in FIG. 14) was identified as having potential benefit.

1. A method for obtaining an aqueously-soluble extract of Sutherlandia frutescens (“Sutherlandia”), the method comprising:
dispersing Sutherlandia plant material in ethanol solution with a concentration of at least 70% v/v;
adjusting the pH of the solution to at least 10;
storing the adjusted solution for at least 24 hours;
re-adjusting the solution to a pH of between 6-7;
storing the re-adjusted solution for at least 24 hours;
separating the supernatant from the sediment, retaining the supernatant; and
filtering the supernatant.

2. The method of claim 1, wherein the ratio of ethanol solution to Sutherlandia plant material is at least 5 ml per gram of extract.

3. The method of claim 1, wherein the ratio of ethanol solution to Sutherlandia plant material is 10 ml per gram of extract.

4. The method of claim 1, wherein the solution is adjusted to a pH of 12.

5. The method of claim 1, wherein the pH is adjusted using sodium hydroxide.

6. The method of claim 1, wherein the pH is re-adjusted using hydrochloric acid.

7. The method of claim 1, wherein the adjusted solution is stored for at least five days.

8. The method of claim 7, wherein the adjusted solution is stored for ten days.

9. The method of claim 1, wherein the re-adjusted solution is stored for at least five days.

10. The method of claim 9, wherein the re-adjusted solution is stored for at least ten days.

11. The method of claim 1, wherein the adjusted solution is mixed daily during storage.

12. The method of claim 1, wherein the re-adjusted solution is mixed for at most the first 50% of the storage period and then left to settle for the remaining period.

13. The method of claim 1, wherein the Sutherlandia plant material is leaf material.

14. The method of claim 1, wherein the method further includes addition of a solubiliser.

15. The method of claim 14, wherein the solubiliser is Kolliphor® HS 15 and/or Kollidon® 25.

16. The method of claim 1, wherein the extract is processed further after extraction to isolate an active fraction.

17. The method of claim 16, wherein the active fraction is isolated to obtain one or more active compounds.

18. (canceled)

19. A composition derived from the method of claim 1.

20. (canceled)

21. The composition of claim 19, wherein the composition is dried.

22. The composition of claim 21, wherein the drying is carried out in combination with a liposomal compound.

23. The composition of claim 19, wherein the composition is further formulated for oral administration using capsules.

24. The composition of claim 19, wherein the composition is formulated for intravenous (IV) injection.

25. The composition of claim 19, wherein the composition is formulated for administration as a cream.

26. The composition of claim 19, wherein the composition is formulated by dilution into a buffered solution.

27. The composition of claim 19, wherein the composition further comprises a known anti-cancer compound.

28. The composition of claim 19, wherein the composition further comprises a known natural or naturally-derived compound.

29. The composition of claim 19, wherein the composition is formulated by drying.

30. A method of treating cancer, comprising administering the composition of claim 19 to a subject in need thereof.

31. Use of the composition of claim 19 in the manufacture of a medicament for the treatment of cancer.
